(19) United States
(12) Patent Application Publication
(10) Pub. No.: US 2019/0104759 A1
(43) Pub. Date: Apr. 11, 2019

(54) NUTRITIONAL COMPOSITIONS AND THEIR USE

(71) Applicant: NESTEC S.A., Vevey (CH)

(72) Inventors: Frederic Destaillats, Servion (CH); Jonas Hauser, Lausanne (CH)

(73) Assignee: NESTEC S.A., Vevey (CH)

(21) Appl. No.: 16/089,124

(22) PCT Filed: Dec. 13, 2016
(86) PCT No.: PCT/EP2016/080792

§ 371 Date: Sep. 27, 2018

Related U.S. Application Data
(60) Provisional application No. 62/315,280, filed on Mar. 30, 2016.
(60) Provisional application No. 62/315,187, filed on Mar. 30, 2016.
(60) Provisional application No. 62/328,047, filed on Apr. 27, 2016.

(51) Int. Cl. A23L 33/12; A23L 33/00; A23C 9/152; A61K 31/20; A61K 31/201; A61K 31/202; A61K 31/23; A61P 25/00

(52) U.S. Cl. CPC A23L 33/12; A23L 33/40; A23C 9/1528; A61K 31/20; A23V 2002/00; A61K 31/202; A61K 31/23; A61P 25/00; A61K 31/201

(57) ABSTRACT

The present invention concerns a nutritional composition to support, to promote, support a health status characterized by optimal brain and cognitive functions' development and/or prevention of neurocognitive deficits. This composition is for use in mammals, preferably in humans, for example in infants.

### FIELD OF THE INVENTION

The present invention concerns a nutritional composition, for example a synthetic nutritional composition, for use to promote and/or support a health status characterized by optimal brain and cognitive functions' development and/or prevention of neurocognitive deficits. This composition is for use in mammals, preferably in humans, more preferably in infants.

### BACKGROUND OF THE INVENTION

During development, especially the first few years of life, children show interesting patterns of neural development and a high degree of neuroplasticity. The relation of brain structure and development and cognitive development is extremely complex and, since the 1990s, a growing area of research.

In a recent study it was demonstrated that the brain structure, in particular the amount and/or temporal-spatial distribution of myelinated matter throughout the brain, of exclusively breastfed infants can differ from infants fed infant formula, and that these differences can be correlated with enhanced intelligence, learning, and/or cognitive functioning in the breastfed infants, in particular in later life (“Breastfeeding and early white matter development: a cross sectional study”, Deoni et al, Neurolmage 82, (2013), 77-86). Said study also clearly demonstrates that there is an association between de novo myelination and brain structure.

The relevance of brain structure, in particular the amount and/or spatial distribution of myelinated matter throughout the brain, for cognitive functioning and intelligence is well documented. Brain structure, in particular the amount and/or spatial distribution of myelin throughout the brain, affects brain connectivity e.g. via what pathway and how quickly and efficiently, messages in the form of neural impulses are communicated within the brain and in particular between different brain regions. This interbrain communication can play a role in cognitive functioning and learning, and may serve to physiological limit/enhance intellectual, cognitive and/or learning potential and to regulate cognitive functioning.

Other studies intend to prove a link between brain growth and cognitive development in infants and particularly in preterm infants or extremely low gestational age newborns (ELGANs) (J. Pediatr. 2009; 155:344-9).

Thus there is a great interest in promoting brain growth and development, particularly in preterm infants, so that to favor their cognitive development.

Accordingly, there is a need to find ways to promote, support or optimize normal cognitive development and function in infants who are born pre-term or with low-birth weight (LBW) or experienced intra-uterine growth retardation (IUGR) or who suffered from growth stunting because of malnutrition, such as suboptimal intra-uterine nutrition, and/or disease.

Previous studies (SvennerholmL. Et al, “Lipid and fatty acid composition of human cerebral myelin during development”, J. Palo (ed.), Myelination and Demyelination, 1978) have demonstrated the presence of certain ethanolamine phosphoglycerides (now known as phosphatidyl ethanolamine) in myelin and white brain matter as being key during early phase of brain maturation.

Accordingly, there is a need to find ways to promote, support or optimise de novo myelination and/or optimal brain and cognitive functions' development in infants who are born pre-term or with low-birth weight (LBW) or experienced intra-uterine growth retardation (IUGR) or who suffered from growth stunting because of malnutrition, such as suboptimal intra-uterine nutrition, and/or disease.

There is more generally a need for nutritional intervention achieving the above mentioned benefits in young mammals, in particular infants and children, preferably infants, but also young pets.

### SUMMARY OF THE INVENTION

The present invention relates to a nutritional composition, for example a synthetic nutritional composition, for infants, in particular for infants born preterm or with low-birth weight (LBW) or who experienced intra-uterine growth retardation (IUGR), such as a pre-term formula or a human milk fortifier. The composition comprises medium chain fatty acid (MCFA) derivatives. Medium chain fatty acids (MCFA) derivatives have been surprisingly found to increase de novo biosynthesis of long chain saturated and/or long chain monounsaturated fatty acids. Such fatty acid derivatives had been previously identified as being fundamental components of brain myelin and white matter and their availability as being key during early phase of brain maturation. Accordingly, by promoting the de novo biosynthesis of long chain saturated and/or long chain monounsaturated fatty acids, the nutritional composition, for example the synthetic nutritional composition, of the present invention, promotes and/or supports a health status characterized by optimal brain and cognitive functions' development and/or prevention of neurocognitive deficits.

Without wishing to be bound by theory, it is hypothized that a larger available pool of acetyl-CoA resulting from dietary FA oxidation is responsible for the observed raised the levels of de novo synthesized palmitic acid and monounsaturated FA.

In one aspect, the present invention provides a nutritional composition, for example the synthetic nutritional composition, comprising one or more medium chain fatty acids (MCFA) derivative to promote and/or support and/or optimize brain and/or cognitive functions' development.

In another aspect, the present invention provides the use of one or more MCFA derivative for the manufacture of a nutritional composition, for example the synthetic nutritional composition, for promoting and/or supporting and/or optimizing brain and/or cognitive functions' development.

In yet another aspect, the present invention provides for a method for promoting and/or supporting and/or optimizing brain and/or cognitive functions' development in an human subject in need thereof comprising administering to such subject a nutritional composition, for example the synthetic nutritional composition, comprising one or more medium chain fatty acids (MCFA) derivative.

In another aspect, the present invention provides for the use of a nutritional composition, for example a synthetic nutritional composition, comprising one or more medium chain fatty acids (MCFA) derivative to promote and/or support and/or optimize brain and/or cognitive functions' development.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 is a schematic representation of fatty acid metabolism wherein variations in specific fatty acids derivatives in RCB-PE compartment (in particular palmitic acid (16:0), vaccenic acid (n-7, 18:1), gondoic acid (n-9, 20:1), erucic acid (n-9, 22:1) and mead acid (n-9, 20:3)) observed in premature infants on consumption of a human milk fortifier according to the present invention are highlighted.

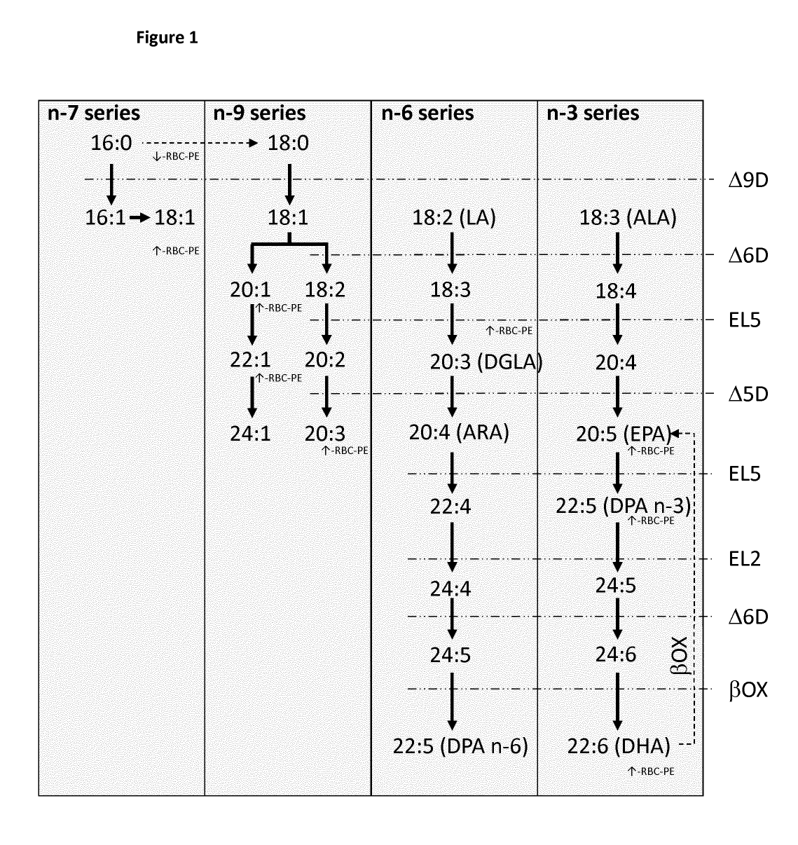

FIG. 2: Shows the impact of DHA on MBP, NF, and/or MBP/NF at day 18 and/or day 30.

FIG. 3: Shows the impact of sialic acid on A2B5, MBP, MAG, NF, MBP/NF, and/or MAG/NF at day 6, day 18 and/or day 30.

FIG. 4: Shows the impact of stearic acid on MAG and MBP mRNA expression and on MBP and BetallI Co-expression.

FIG. 5: Shows the impact of DHA on MAG and MBP mRNA expression and on MBP and BetallI Co-expression.

### DETAILED DESCRIPTION OF THE INVENTION

Definitions

As used herein, the following terms have the following meanings.

Within the context of the present invention, the term “promote” and/or “promoting” indicates a factor or a number of factors causing a certain process to occur.

Within the context of the present invention, the term “support” and/or “supporting” indicates a factor or a number of factors sustaining a certain process once it has started to occur.

The term “optimise” as used herein refers to an improvement or enhancement.

The expression “brain and cognitive functions' development” within the context of the present invention in meant to identify a normal development for brain and/or mental processes, structures, skills and abilities selected, for example, in the group consisting of: de novo myelination; brain structure, in particular the amount and spatial distribution of myelinated matter throughout the brain, and/or in specific brain regions; brain connectivity; intellectual potential; cognitive potential; learning potential; cognitive functioning; cognition; cognitive skills and abilities; and/or learning.

The term “de novo myelination” as used herein refers to development myelination and in particular the process by which naked axons in the brain of a subject are myelinated during growth and development. It is a process that starts, in particular in specific brain regions, in utero and continues postnatally, and that is most prolific in the first 5 years of a human subject's life, in particular the first 2 & 3 years of a human's life, in particular the first years of human's life.

The term “cognition” as used herein refers to the intellectual processes by which one individual becomes aware of, perceives, or comprehends ideas; thus, the ability to think and understand. Cognition includes all aspects of information processing, perception, attention, thinking, reasoning, understanding and remembering as well as psychomotor, language, memory, concentration, executive functions and problem-solving abilities.

The term “cognitive skills” or “cognitive abilities” as used herein refer to cognitive and/or mental ability or capacity of a subject. In particular the term may refer to one or more of; information processing, perception, attention, thinking, reasoning, understanding and remembering, psychomotor including gross motor and fine motor potential, visual including visual reception, language including expressive and receptive language, memory and recall, concentration, executive function including problem-solving, decision-making and inhibition.

The term “brain structure” as used herein refers to the structure of grey and white matter within the brain and specific brain regions, and in particular to myelinated white matter within the brain and specific brain regions as determined by de novo myelination i.e. by the de novo structural deposition of myelin. More particularly the term refers to the amount and/or spatial distribution of myelinated matter throughout the brain, and/or in specific brain regions, and even more particularly the amount and/or temporal spatial distribution of myelinated matter throughout the brain and/or in specific brain regions.

The term “intellectual potential” as used herein refers to the possible intellectual ability or capacity attainable by a subject as determined by physiological factors. In particular intellectual potential may refer to fluid intelligence.

The term “fluid intelligence” as used herein refers to a subject's neural potential and/or a subject's novel or abstract problem solving capability as determined by physiological factors. This is distinct from crystallized intelligence which is determined, at least in part by learned or acculturated knowledge.

The term “cognitive potential” as used herein refers to the possible cognitive and/or mental ability or capacity possibly attainable by a subject as determined by physiological factors. In particular the term may refer to one or more of; information processing potential, perception potential, attention potential, thinking potential, reasoning potential, understanding and remembering potential, psychomotor potential including gross motor and fine motor potential, visual potential including visual reception potential, auditory potential, language potential including expressive and receptive language potential, memory and recall potential, concentration potential, executive function potential including problem-solving, decision-making and inhibition potential.

The term “learning potential” as used herein refers to the possible ability or capacity a subject has to learn e.g. how easily and/or quickly a subject may be able to acquire knowledge or skills through experience, study or being taught, as determined by physiological factors. As well as the possible ability a subject has to adapt in response to environmental factors, as determined by physiological factors.

The term “Learning” as used herein refers to the acquisition of knowledge or skills through experience, study, or by being taught.

In promoting, supporting or optimising cognitive potential, learning potential and/or intellectual potential, the compositions of the invention may have a short term or long term effect on cognitive functioning, including the development of cognitive functions, and/or learning, and on preventing or minimising any neuro cognitive deficits, impairment or delay.

Said short term effect may only be apparent in days, weeks, or months.

Said long term effect may only be apparent in years e.g. 1, 2, 3, 4, 5, 6, 7, 8, 9, 10, 20, 30, 40, 50, 60. 70, 80, 90 years.

In an embodiment of the invention cognitive potential is selected from the group consisting of vision potential, auditory potential, motor function and psychomotor potential (including coordination and execution of movement potential), and/or executive functioning potential including problem solving potential, social processing, behaviour interaction potential, and social-emotional functioning potential.

In promoting supporting or optimising vision potential, auditory potential, motor function and psychomotor potential, and/or executive functioning potential including problem solving potential, social processing potential, behaviour interaction potential, and/or language potential, the compositions of the invention may have a short term or long term effect e.g. enhancement effect, on vision, motor function and psychomotor function, and/or executive functioning including problem solving, social processing, behaviour interaction, and/or language. Said short term effect may only be apparent in days, weeks, or months.

Said long term effect may only be apparent in years e.g. 1, 2, 3, 4, 5, 6, 7, 8, 9, 10, 20, 30, 40, 50, 60. 70, 80, 90 years.

The term “subject” as used herein refers to a mammal, in particular a cat, dog or human, more particularly the term refers to a human, even more particularly a human infant or child and even more particularly still a human infant or child fed infant formula and/or growing up milk.

The term “infant” as used herein refers to a human infant of up to 12 months of age and includes preterm and very preterm born infants, infants having a low birth weight i.e. a new born having a body weight below 2500g (5.5 pounds) either because of preterm birth or restricted fetal growth, and infants born small for gestational age (SGA) i.e. babies with birth weights below the 10th percentile for babies of the same gestational age.

The term “child” as used herein refers to a human of 1 to 18 years of age, more specifically a human of 1 to 10 years of age, even more specifically a human of 1 to 5 years of age, and even more specifically a human of 1 to 2 years of age.

The term “formula fed infant or child” as used herein refers to an infant or child fed either infant formula and/or growing up milk.

The term “breastfed subject” as used herein refers to a subject, In particular an infant or child, fed human breastmilk, in particular from a nutritionally replete mother.

A “preterm” or “premature” means an infant or young child that was not born at term. Generally it refers to an infant born prior to the completion of 37 weeks of gestation.

The expression “Term born infant” indicates an infant born after 37 weeks gestation.

Within the context of the present invention, the term “Low birth weight” indicates a newborn's body weight below 2500g (5.5 pounds), either as a result of preterm birth (i.e. before 37 weeks of gestation) and/or due to restricted foetal growth.

By the expression “low birth weight”, it should be understood as any body weight under 2500g at birth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

Within the context of the present invention, the term “Small-for-gestational-age (SGA)” refers to babies with birth weights below the 10th percentile for babies of the same gestational age.

The expression “Postnatal period” is the period beginning immediately after the birth of a child and extending for about six weeks.

The expression “nutritional composition” means a composition which nourishes a subject. This nutritional composition is usually to be taken enterally, orally, parenterally or intravenously, and it usually includes a lipid or fat source and optionally a protein source and/or optionally a carbohydrate source and/or optionally minerals and vitamins.

Preferably, the nutritional composition is for oral use.

The expression “hypoallergenic nutritional composition” means a nutritional composition which is unlikely to cause allergic reactions.

The expression “synthetic composition” means a mixture obtained by chemical and/or biological means, which can be chemically identical to the mixture naturally occurring in mammalian milks.

The expression “synthetic nutritional composition” identifies nutritional composition as above defined which are obtained by chemical and/or biological means, which can be chemically identical to a the mixture which also naturally occurr, for example in mammalian milks. As detailed in Example 1, synthetic nutritional compositions as herein defined are comprised within the scope of the present invention and all the embodiments described in the present application apply as well to such synthetic nutritional composition.

In an embodiment said synthetic nutritional composition is selected from the group consisting of; growing up milk, infant formula or a composition for infants that is intended to be added or diluted with human breast milk (hereinafter “HM”) e.g. HM fortifier, or a food stuff intended for consumption by an infant and/or child either alone or in combination with HM e.g. complementary foods.

The expression “infant formula” means a foodstuff intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae).

The expression “starter infant formula” means a foodstuff intended for particular nutritional use by infants during the first four months of life.

The expression “pre-term formula” or “preterm formula” means an infant formula intended for a preterm infant or for an infant with low-birth weight (LBW) or who experienced intra-uterine growth retardation (IUGR) or for infants small for gestational age (SGA).

The expression “fortifier” or “human milk fortifier” (HMF) refers to liquid or solid nutritional compositions suitable for mixing with breast milk or infant formula, for example a preterm infant formula. By the term “milk fortifier”, it is meant any composition used to fortify or supplement either human breast milk, infant formula, growing-up milk or human breast milk fortified with other nutrients. The term “fortifier” refers to a composition which comprises one or more nutrients having a nutritional benefit for infants, both preterm infants, with low-birth weight (LBW) or infants who experienced intra-uterine growth retardation (IUGR) or infants small for gestational age (SGA), and term infants.

The term “weaning period” means the period during which the mother's milk is substituted by other food in the diet of an infant.

The “mother's milk” should be understood as the breast milk or colostrum of the mother (=Human Breast Milk=HBM).

The term “fatty acid” as used herein indicates a carboxylic acid with a long aliphatic chain, which is either saturated or unsaturated and refers to a compound of formula (XII)

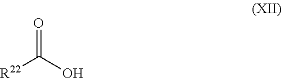

Wherein

R²² is a C3 to C43 branched or unbranched acyclic alkyl, or acyclic alkenyl group. More particularly, R²² is a C3 to C43 branched or unbranched acyclic alkyl, or acyclic alkenyl group, and even more particularly a C3 to C 28 branched or unbranched acyclic alkyl, or acyclic alkenyl group. Mixture of such compounds are also comprised within the scope of the invention and/or the term.

The term “medium chain fatty acid” (MCFA) as used within the context of the present invention identifies a fatty acid as above defined wherein R²² is C₇ or C₉ branched or unbranched acyclic alkyl, or acyclic alkenyl group. Non limiting examples of such MCFA are: capric acid (8:0) and caprylic acid (10:0). Mixture of such compounds are also comprised within the scope of the invention and/or the term.

The term “long chain fatty acid” (LCFA) as used within the context of the present invention identifies a fatty acid as above defined wherein R²² is C₁₁ branched or unbranched acyclic alkyl, or acyclic alkenyl group or longer, in particular C₁₃ to C₂₃. Long chain fatty acids may be saturated, mono unsaturated (MUFA) or polyunsaturated (PUFA). Non limiting examples of such LCFA are: lauric acid (12:0), myristic acid (14:0), palmitic acid (16:0), stearic acid (18:0), arachidic acid (20:0), behenic acid (22:0), lignoceric acid (24:0), vaccenic acid (n-7, 18:1), gondoic acid (n-9, 20:1), erucic acid (n-9, 22:1), mead acid (n-9, 20:3), alpha-linolenic acid (ALA) (n-3, 18:3), Eicosapentaenoic acid (EPA) (n-3, 20:5), Docosapentaenoic acid (DPA n-3) (n-3, 22:5), Docosahexaenoic (DHA) (n-3, 22:6), Linoleic acid (LA) (n-6, 18:2), Dihomo-gamma-linolenic acid (DGLA) (n-6, 20:3), Arachidonic acid (AA or ARA) (n-6, 20:4), and Docosapentaenoic acid (DPA n-6) (n-6, 22:5). Long chain fatty acids are typically product of fatty acid metabolism in humans. LCFA belonging to the n-6 and n-3 series constitute the so called “essential fatty acids” whose biosynthesis can't be initiated by metabolic mechanisms in the absence of linoleic and alpha-linoleic acid substrate introduced with the diet.

Long chain fatty acids of the n-7 and n-9 series are on the other hand often defined as being “non-essential” as they can biosynthetized de novo.

Mixture of such compounds are also comprised within the scope of the invention and/or the term.

The term “short chain fatty acid” (SCFA) as used within the context of the present invention identifies a fatty acid as above defined wherein R²² is C₃ to C₅ branched or unbranched acyclic alkyl, or acyclic alkenyl group or shorter. Non limiting examples of such SCFA are: butyric acid (4:0), and caproic acid (6:0).

Mixture of such compounds are also comprised within the scope of the invention and/or the term.

The term “de novo biosynthesis of fatty acids derivatives” within the context of the present invention refers to the ability of mammals, in particular humans, to synthetize fatty acids derivatives by metabolic processes starting from available carbon-containing substrates (such as for example amino acids, carbohydrates, fatty acids).

The term “fatty acid derivative” as used herein refers to a compound comprising a fatty acid, other than a phospholipid, and in particular to a free fatty acid, and/or a monoacylglycerol (hereinafter MAG), and/or a diacylglycerol (hereinafter DAG), and/or a triacylgylcerol (hereinafter TAG) and/or a cholesterol ester. More particularly the term refers to a MAG, DAG, TAG and/or a cholesterol ester. Even more particularly the term refers to a TAG.

Mixture of such compounds are also comprised within the scope of the invention and/or the term.

The term “MAG” as used herein refers to a glycerol molecule in which one of the OH groups has formed an ester bond with a fatty acid. In particular the term “MAG” as used herein refers to a compound of formula (X)

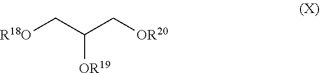

Wherein,

two of R¹⁸R^{19 or} R²⁰ are H and wherein one of R¹⁸R^{19 or} R¹⁹ or R²⁰ is a C4 to C44 saturated or unsaturated acyl group.

Mixture of such compounds are also comprised within the scope of the invention and/or the term.

The term “DAG” as used herein refers to glycerol molecule in which two of the OH groups have formed an ester bond with two fatty acids. In particular the term “DAG” as used herein refers to a compound of formula (X)

Wherein,

one of R¹⁸R¹⁹ or R²⁰ are H and wherein two of R¹⁸R^{19 or} R²⁰ are C4 to C44 saturated or unsaturated acyl groups. The two C4 to C44 saturated or unsaturated acyl groups may be the same or different.

Mixture of such compounds are also comprised within the scope of the invention and/or the term.

The term “TAG” as used herein refers to glycerol molecule in which three of the OH groups have formed an ester bond with three fatty acids. In particular the term “TAG” as used herein refers to a compound of formula (X)

Wherein,

Wherein all R¹⁸R^{19 or} R²⁰ are C4 to C44 saturated or unsaturated acyl groups. The three C4 to C44 saturated or unsaturated acyl groups may all be the same, all different, or two may be the same and one different. Mixture of such compounds are also comprised within the scope of the invention and/or the term.

The term “cholesterol ester” as used herein refers to a compound of formula (XI)

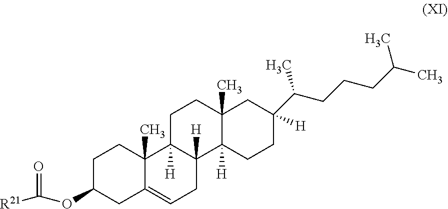

Wherein,

R²¹ is a C2 to C43 branched or unbranched acyclic alky, or acyclic alkenyl group. Mixture of such compounds are also comprised within the scope of the invention and/or the term.

Within the context of the present invention, the term “Phosphatidylethanolamine” indicates a compound of formula (VII)

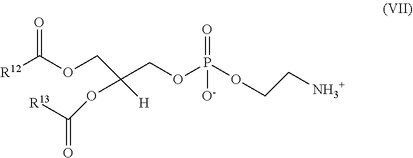

Wherein R¹²is a C3 to C43 branched or unbranched acyclic alkyl, or acyclic alkenyl group and,

R¹³ is a C3 to C43 branched or unbranched acyclic alkyl, or acyclic alkenyl group. More particularly, R¹² and R¹³ are, independently of each other, C7 to C27 branched or unbranched acyclic alkyl, or acyclic alkenyl groups which together with their adjacent carbonyl group correspond to C8 to C28 saturated or unsaturated fatty acid residues, and even more particularly R¹² and R¹³ are, independently of each other, C13 to C23 branched or unbranched acyclic alkyl, or acyclic alkenyl groups which together with their adjacent carbonyl group correspond to C14 to C24 saturated or unsaturated fatty acid residues. Mixture of such compounds are also comprised within the scope of the invention and/or the term.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “vitamin” as used herein refers to any vitamin. Non limiting examples of vitamins include: vitamin A, vitamin B1, vitamin B2, vitamin B6, vitamin K, vitamin C, vitamin D, niacin, biotin, pantothenic acid, folic acid, vitamin B12, and combinations thereof.

Within the context of the present invention, the term “folic acid” is to be intended as identifying all the folic acid present in the nutritional compositions, for example synthetic nutritional compositions, of the invention either as such or in the form of one physiologically acceptable salt thereof (folate) and mixtures thereof.

All percentages are by weight unless otherwise stated.

The invention will now be described in further details. It is noted that the various aspects, features, examples and embodiments described in the present application may be compatible and/or combined together.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

The terms “in particular” or “more particularly” as used herein should not be considered limiting but should be interpreted as being synonymous with “for example” or “especially”.

### Embodiments

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

In one embodiment, Nutritional compositions according to the present invention, for example synthetic nutritional compositions, provide MCFA in the form of TAGS.

In one embodiment, nutritional compositions according to the present invention, for example synthetic nutritional compositions, promote and/or support and/or optimizing brain and/or cognitive functions' development via promotion and/or support and/or optimization of de novo biosynthesis of long chain saturated and/or long chain monounsaturated fatty acid derivatives.

In one embodiment, nutritional compositions according to the present invention, for example synthetic nutritional compositions promote and/or support and/or optimizing brain and/or cognitive functions' development via promotion and/or support and/or optimization of de novo biosynthesis of long chain saturated and/or long chain monounsaturated fatty acid derivatives and/or via promotion and/or support and/or optimization of brain de novo myelination.

In another embodiment, the present invention provides for the use of a nutritional composition, for example a synthetic nutritional composition, comprising one or more medium chain fatty acids (MCFA) derivative to promote and/or support and/or optimize de novo biosynthesis of long chain saturated and/or long chain monounsaturated fatty acids.

In another embodiment, the present invention provides for the use of a nutritional composition, for example a synthetic nutritional composition, comprising one or more medium chain fatty acids (MCFA) derivative to promote and/or support and/or optimize brain de novo myelination.

In another embodiment, the use of a nutritional compositions according to the present invention, for example a synthetic nutritional composition, is provided to promote and/or support and/or optimizing brain and/or cognitive functions' development via promotion and/or support and/or optimization of de novo biosynthesis of long chain saturated and/or long chain monounsaturated fatty acid derivatives and/or via promotion and/or support and/or optimization of brain de novo myelination.

In another embodiment, the use of a nutritional compositions according to the present invention, for example a synthetic nutritional composition, is provided to promote and/or support and/or optimizing brain and/or cognitive functions' development via promotion and/or support and/or optimization of de novo biosynthesis of long chain saturated and/or long chain monounsaturated fatty acid derivatives and/or via promotion and/or support and/or optimization of brain de novo myelination, in an infant who was born preterm or with low-birth weight (LBW) or who experienced intra-uterine growth retardation (IUGR).

In one embodiment, nutritional compositions according to the present invention, for example synthetic nutritional compositions, promote and/or support and/or optimizing brain and/or cognitive functions' development in an infant who was born preterm or with low-birth weight (LBW) or who experienced intra-uterine growth retardation (IUGR).

In one embodiment, the nutritional composition according to the present invention, for example synthetic nutritional compositions, promote and/or support and/or optimizing brain and/or cognitive functions' development and prevent of neurocognitive deficits.

The composition of the invention may be any type of composition suitable and intended for direct administration to a subject, for example an infant who was born preterm or with low-birth weight (LBW) or who experienced intra-uterine growth retardation (IUGR).

In particular the composition will be a synthetic nutritional composition.

Preterm Infant Formula

In one embodiment, nutritional compositions according to the present invention is a pre-term formula.

In one embodiment, the pre-term formula according to the present invention comprises MCFA in an amount of up to 40% by weight of the total content of lipid.

In an embodiment of the invention, the preterm formula comprises at least 20% MCT by weight of the total lipid content, such as at least 25%, preferably at least 30%, such as at least 35%, even more preferably 40% by weight of the total lipid content.

In one embodiment, the preterm formula according to the present invention comprises MCFA derivatives in amount ranging from 0.1 to 25% w/w, for example in an amount ranging from 0.5 to 20% w/w, for example in an amount ranging from 1 to 15% w/w of dry powder.

In another embodiment, the liquid preterm formula according to the present invention comprises MCFA derivatives in amount ranging from 0.01 to 4 g/100 mL of liquid formula, for example in an amount ranging from 0.05 to 3 g/100 mL, for example in an amount ranging from 0.1 to 3.5 g/100 mL.

In another embodiment, the preterm formula according to the present invention comprises MCFA derivatives in amount ranging from 0.01 to 5 g/100 Kcal of formula, for example in an amount ranging from 0.05 to 4 g/100 Kcal, for example in an amount ranging from 0.1 to 3 g/100 Kcal.

In one embodiment, the preterm formula according to the present invention comprises fatty acid derivatives in amount ranging from 10 to 40% w/w, MCFA derivatives in amount ranging from 0.1 to 25% w/w, 5 to 50% w/w protein and 10 to 80% w/w carbohydrates.

Human Milk Fortifier

In one embodiment, the nutritional compositions according to the present invention is a human milk fortifier. In one embodiment, the human milk fortifier according to the present invention comprises MCFA derivatives in amount ranging from 2 to 40% w/w, for example in an amount ranging from 5 to 30% w/w, for example in an amount ranging from 5 to 20% w/w for example in an amount ranging from 7 to 18% w/w.

In another embodiment, the human milk fortifier according to the present invention comprises MCFA derivatives in amount ranging from 0.08 to 1.6 g/100 mL of HMF reconstituted in human breast milk, for example in an amount ranging from 0.2 to 1.2 g/100 mL, for example in an amount ranging from 0.25 to 0.75 g/100 mL.

In another embodiment, the human milk fortifier according to the present invention comprises MCFA derivatives in amount ranging from 2 to 10 g/100 Kcal of HMF, for example in an amount ranging from 1.2 to 7.5 g/100 Kcal, for example in an amount ranging from 1.75 to 4.5 g/100 Kcal.

In another embodiment, the human milk fortifier according to the present invention comprises MCFA derivatives in amount ranging from 0.05 to 2.5 g/100 Kcal of HMF reconstituted in human breast milk, for example in an amount ranging from 0.2 to 2.0 g/100 Kcal, for example in an amount ranging from 0.5 to 1.5 g/100 Kcal.

In one embodiment, the human milk fortifier according to the present invention comprises MCFA derivatives in amount ranging from 40 to 80% w/w of total fatty acid derivatives, for example in an amount ranging from 50 to 75% w/w, for example in an amount ranging from 55 to 70% w/w.

In another embodiment, the human milk fortifier according to the present invention comprises MCFA derivatives in amount ranging from 5 to 40% w/w of total fatty acid derivatives/100 mL of HMF reconstituted in human breast milk, for example in an amount ranging from 10 to 20% w/w of total fatty acid derivatives/100 mL of HMF reconstituted in human breast milk.

In another embodiment, the human milk fortifier according to the present invention comprises MCFA derivatives in amount ranging from 5 to 40% w/w of total fatty acid derivatives/100 Kcal of HMF reconstituted in human breast milk, for example in an amount ranging from 10 to 20% w/w of total fatty acid derivatives/100 Kcal of HMF reconstituted in human breast milk.

In one embodiment, the human milk fortifier according to the present invention comprises 5 to 40% w/w fatty acid derivatives, wherein 40 to 80% w/w are constituted by MCFA derivatives.

In one embodiment, the human milk fortifier according to the present invention comprises 5 to 30% w/w fatty acid derivatives, wherein 50 to 75% w/w are constituted by MCFA derivatives, 20 to 50% w/w protein and 15 to 40% w/w carbohydrates.

### Other Ingredients

The nutritional composition, according to the present invention, for example the synthetic nutritional composition, can besides from comprising MCFA derivatives comprise other nutrients, such as e.g. lipids (including fatty acid derivatives), proteins, carbohydrates, vitamins, minerals, probiotics, or prebiotics.

### Lipids

In the context of the present invention, the term “lipid” refers to one or more lipids and may be any free fatty acid or ester of fatty acids that are suitable for being fed to an infant. Lipid includes for example monoglycerides, diglycerides, triglycerides, phospholipids, cholesterol, free fatty acids, derivatives of fatty acids and combinations thereof.

The lipids used to prepare the fortifier can be naturally liquid or solid at room temperature. In some particular embodiments at least a part of the lipids used to prepare the fortifier are naturally liquid at room temperature.

In an embodiment of the present invention, the nutritional composition, for example the HMF according to the invention, comprises lipid in an amount above 25% of the caloric content.

In another embodiment, the nutritional composition, for example the HMF according to the invention, comprises lipid in an amount above 75% of the caloric content.

In some embodiments of the invention, lipids are present in the nutritional composition, for example the HMF, in an amount of at least 30% of the caloric content, such as at least 35% of the caloric content.

In an embodiment of the invention, the lipids are selected from the group of monoglycerides, diglycerides, triglycerides, phospholipids, cholesterol, free fatty acids, derivatives of fatty acids and combinations thereof.

In a particular embodiment of the invention, the lipids are selected from the group of arachidonic acid, docosahexaenoic acid, eicosapentaenoic acid, linoleic acid, α-linolenic acid, milk fat, structured lipids phospholipid, and combinations thereof. Structured lipids may be monoglycerides, diglycerides, triglycerides, cholesterol, palmitic acid esterified in the sn-2 position or interesterified palm stearin.

Lipids may be derived from various sources. The lipid source may be any lipid or fat source which is suitable for use in nutritional compositions, to be fed to infants, for example some vegetable or animal fats or oils.

In an embodiment of the invention, the lipid is provided from oils or fats.

Preferred lipid sources include coconut oil, soy oil, corn oil, olive oil, safflower oil, sunflower oil, palm oil, palm kernel oil, low erucic rapeseed oil (canola oil), marine oil, cottonseed oil, soy lecithin's, palm oil, milk fat, structured lipids, egg-derived oils, fungal oils, algal oils and combinations thereof. Particularly preferred oils are canola oils, soy lecithin, palm olein, and sunflower oil.

Dietary lipids are essential for an infant since they provide the infant with much of his energy needs, such as the essential polyunsaturated fatty acids and lipid soluble vitamins.

The amount and composition of dietary lipids affect both the growth pattern and the body composition of the infant.

In an embodiment of the invention, the lipid comprises one or more polyunsaturated fatty acid, preferably long chained polyunsaturated fatty acids.

The polyunsaturated fatty acids, and in particular the long chain ones are important for the cell membrane function and the development of the brain and visual system in infants. Further, the long chain polyunsaturated fatty acids are important in the formation of bioactive eicosanoids. Brain grey matter and the retina are complex neural functions related to energy supply and the composition of dietary fatty acids.

In a particular embodiment of the invention, the composition comprises arachidonic acid, docosahexaenoic acid, or a combination thereof as the lipid component. The arachidonic acid and docosahexaenoic acid may be alone or in combination with other lipids, such as linoleic acid and/or α-linolenic acid.

In one embodiment, the content of arachidonic acid in the nutritional composition according to the invention, for example a HMF, is at least 0.005% w/w, such as at least 0,0075%, for example at least 0.01% w/w.

In one embodiment, the content of arachidonic acid in the nutritional composition of the invention, for example a preterm formula, ranges between 0.001% w/w to 1% w/w , for example from 0.01% w/w to 0.5% w/w.

In one embodiment, the content of arachidonic acid in the HMF according to the present invention is at least 0.2% by weight of the total lipid content, such as at least 0,30%, in particular at least 0,38%, even more preferably at least 0.65%, such as 0,70% by weight of total lipid content.

In another embodiment the HMF comprises arachidonic acid in an amount of up to 2.5% by weight on the total lipid content, such as at in the range of 0.2 to 2.0%, preferably from 0.3 to 1.5%, such as from 0.35 to 1.2%, even more preferably from 0.4 to 0.9% by weight of the total lipid content.

In one embodiment, the content of docosahexaenoic acid in the nutritional composition of the invention, for example a preterm formula, ranges between 0.001% w/w to 1% w/w , for example from 0.01% w/w to 0.5% w/w.

In one embodiment, the content of docosahexaenoic acid in the nutritional composition according to the invention, for example a HMF, is at least 0.05% w/w, such as at least 0.075% w/w, for example at least 0.1% w/w.

In one embodiment, the content of docosahexaenoic acid in the HMF according to the present invention is ranging from 0.05% to 5% w/w, such as from 0.075% to 3% w/w, for example from 0.1% to 2% w/w.

In one embodiment, the content of docosahexaenoic acid in the HMF according to the present invention is preferably at least 0.05% by weight of the total lipid content, such as at least 0,1%, for example at least 0.15%, such as 0,5% by weight of total lipid content.

In another specific embodiment the composition comprises docosahexaenoic acid in an amount of up to 3.0% by weight on the total lipid content, such as from 0.0.5% to 2.5%, preferably from 0,1 to 2.0%, such as from 0.15 to 1.50%by weight of the total lipid content.

In on embodiment, if the nutritional composition according to the present invention, comprises fatty acid derivatives comprising ARA and DHA, said ingredients may for example be comprised in the composition of the invention in amounts resulting in a weight ratio of DHA:ARA in the range of 4:1 to 1:4, for example 3:1 to 1:3, for example 2:1 to 1:2, for example 1.5:1 to 1:1.5, in particular 1.1:1 to 1:1.1.

Docosahexaenoic (DHA) and arachidonic acid (ARA) are both known to provide beneficial effects in infants, such as enhancing brain and vision development. DHA and ARA are therefore necessary for infants, both preterm and term infants, but in particular for a preterm infant. Further, DHA and ARA have shown beneficial effects on measures of cognitive development during the first year of life, and on immune phenotypes.

Non-limiting examples of suitable sources of ARA and DHA include marine oil, egg-derived oils, fungal oil, algal oil, and combinations thereof.

In still another embodiment of the invention, the nutritional composition according to the invention, for example a synthetic nutritional composition, comprises linoleic acid, α-linolenic acid or a combination thereof as lipid.

In a specific embodiment of the invention, the nutritional composition of the invention, for example a human milk fortifier, comprises linoleic acid in an amount ranging from 0.1% w/w to 5% w/w of dry composition.

In another embodiment of the invention, the nutritional composition of the invention, for example a preterm powder formula, comprises linoleic acid in an amount ranging from 0.5 to 10% w/w of dry composition.

In another embodiment of the invention, the nutritional composition of the invention, for example a preterm liquid formula, comprises linoleic acid in an amount ranging from 0.05 to 5 g/100 mL of formula.

In a specific embodiment of the invention, the nutritional composition of the invention, for example a human milk fortifier, comprises α-linolenic acid in an amount ranging from 0.1% w/w to 3% w/w of dry composition.

In another embodiment of the invention, the nutritional composition of the invention, for example a preterm powder formula, comprises α-linolenic acid in an amount ranging from 0.01 to 5% w/w of dry composition.

In another embodiment of the invention, the nutritional composition of the invention, for example a preterm liquid formula, comprises α-linolenic acid in an amount ranging from 0.01 to 2 g/100 mL of formula.

The lipid may also be eicosapentaenoic acid (20:5n-3).

In a specific embodiment of the invention, the nutritional composition of the invention, for example a human milk fortifier, comprises eicosapentaenoic acid in an amount ranging from 0.01% w/w to 5% w/w of dry composition.

In another embodiment of the invention, the nutritional composition of the invention, for example a preterm powder formula, comprises eicosapentaenoic acid in an amount ranging from 0.01 to 5% w/w of dry composition.

In another embodiment of the invention, the nutritional composition of the invention, for example a preterm liquid formula, comprises eicosapentaenoic acid in an amount ranging from 0.05 to 20 mg/100 mL of formula.

In an embodiment of the invention, the lipid comprises one or more of phospholipids.

In one embodiment, the content of phospholipid in the composition according to the present invention, for example a human milk fortifier, is preferably from 0.5 to 20% by weight of the total lipid content, such as from 0.8 to 15%, even more preferably from 1.0 to 10%, such as from 1.5 to 8% by weight of the total content of lipid.

In one embodiment, phospholipids may be phosphatidylcholine, phosphatidylserine, phosphatidylinositol and/or sphingomyelin, in particular sphingomyelin.

However in a particular embodiment of the invention, the composition according to the present invention does not comprise any phospholipids.

Additional Ingredients

The compositions of the invention can also comprise any other ingredients or excipients known to be employed in the type of composition in question e.g. infant formula, preterm formula and/or human milk fortifiers.

Non limiting examples of such ingredients include: proteins, amino acids, carbohydrates, oligosaccharides, lipids, prebiotics or probiotics, nucleotides, nucleosides, other vitamins, minerals and other micronutrients.

### Vitamins:

The composition according to the present invention may further comprise one or more vitamin. The presence and amounts of specific minerals and other vitamins will vary depending on the intended population.

In one embodiment, vitamins may be folic acid, vitamin B12 and vitamin B6, in particular folic acid and vitamin B12, in particular folic acid.

In one embodiment of the invention, the composition comprises one or more vitamin which is lipid-soluble, for example one or more of vitamin A, vitamin D, vitamin E and vitamin K.

Vitamin D is important for supporting a large number of physiological processes such as neuromuscular function and bone mineralisation. The preferred amount of vitamin D given to an infant in the first months of life is 800-1000 IU per day, i.e. 20-25 μg per day.

Only small amounts of vitamin D are transported to the breast milk. Thus, human breast milk contains low amounts of vitamin D. An infant who is breast fed therefore will need an additional supply of vitamin D. There is therefore a need for a nutritional composition, for example a synthetic nutritional composition, to supply energy to an infant which also contributes to the recommended intakes of vitamin D.

An infant is normally fed 5-8 times a day, and the amount of vitamin per serving should therefore not exceed 5.0 μg vitamin D, preferably the amount per serving should be 3-4 μg vitamin D.

In one embodiment, the amount of vitamin D in the nutritional composition, in particular a human milk fortifier, is thus preferably from 75 to 125 μg per 100 g of the total composition, such as from 80 to 120 μg per 100 g of the total composition, even more preferably from 85 to 110 μg per 100 g of the total composition.

In an embodiment of the invention, the composition comprises from 0.5 to 10.0 μg vitamin D per 100 kcal of the composition, such as from 1.0 to 8.0 μg vitamin D per 100 kcal, preferably from 2.0 to 7.0 μg vitamin D per 100 kcal, even more preferably from 3.5 to 5.5 μg vitamin D per kcal of the composition.

Vitamin K is important to help blood to clot. The human breast milk contains low amounts of vitamin K and the infants immature intestinal tract may not produce enough vitamin K to meet the infants own needs.

In one embodiment, he amount of vitamin K in the nutritional composition according to the present invention, for example a human milk fortifier, is preferably from 50 to 400 μg per 100 g of the total composition, such as from 100 to 300 μg per 100 g of the total composition, preferably 200 μg per 100 g of the total composition.

In an embodiment of the invention, the nutritional composition, comprises from 1 to 30 μg vitamin K per 100 kcal, such as form 5 to 20 μg vitamin K per 100 kcal, preferably from 7 to 15 μg vitamin K per 100 kcal, even more preferably from 8 to 12 μg vitamin K per 100 kcal.

Vitamin A prevents infections, while vitamin E protects the body from harmful substances and serves as an antioxidant. The daily intake of vitamin A in an infant is preferably from 400 to 1000 μg/kg/day.

Thus, in an embodiment of the invention, the nutritional composition of the invention, for example a human milk fortifier, comprises from 1 to 30 mg vitamin A per 100 g of the total composition, such as from 5 to 20 mg per 100 g of the total composition, preferably from 8 to 15 mg per 100 g of the total composition.

In an embodiment of the invention, the composition comprises from 0.1 to 3.0 mg vitamin A per 100 kcal, such as from 0.2 to 2.0 mg vitamin A per 100 kcal, preferably from 0.3 to 1.2 mg vitamin A per 100 kcal, even more preferably from 0.4 to 0.8 mg vitamin A per 100 kcal.

The daily intake of vitamin E in an infant is preferably 2.2 to 11 mg per day. Thus, in an embodiment of the invention, the nutritional composition of the invention, in particular a human milk fortifier, comprises from 50 to 200 mg vitamin E per 100 g of the total composition, such as from 75 to 150 mg vitamin E per 100 g of the total composition, preferably from 85 to 115 mg vitamin E per 100 g of the total composition.

In an embodiment of the invention, the composition comprises from 1 to 10.0 mg vitamin E per 100 kcal, such as from 2 to 8.0 mg vitamin E per 100 kcal, preferably from 3 to 7 mg vitamin E per 100 kcal, even more preferably from 4 to 6 mg vitamin E per 100 kcal.

Minerals:

In an embodiment of the invention, the composition further comprises one or more mineral.

Examples of minerals are sodium, potassium, chloride, calcium, phosphate, magnesium, iron, zinc, copper, selenium, manganese, fluoride, iodine, chromium, or molybdenum. The minerals are usually added in salt form.

The minerals may be added alone or in combination.

In on embodiment, minerals may be iron, zinc, calcium, phosphorus, copper, and magnesium, in particular iron.

In a specific embodiment of the invention, the mineral is calcium.

Protein:

In another embodiment of the invention the composition further comprises a protein source. The composition may comprise one or more protein.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. The proteins can be at least partially hydrolyzed in order to enhancement of oral tolerance to allergens, especially food allergens. In that case the composition is a hypoallergenic composition.

In one embodiment, the nutritional composition according to the invention may be cow's milk whey based infant formula. The formula may also be a hypoallergenic (HA) formula in which the cow milk proteins are (partially or extensively) hydrolysed. The formula may also be based on soy milk or a non-allergenic formula, for example one based on free amino acids.

In an embodiment of the invention, the nutritional composition, for example a human milk fortifier, comprises up to 55% protein of the caloric content, for example up to 50%.

In a preferred embodiment of the invention, the composition comprises up to 45% protein, such as up to 40% protein, or up to 35% protein, based on the caloric content.

In another embodiment of the invention, the composition is free of protein. By “free” is hereby meant that the composition may comprise traceable amounts of protein, such as less than 1% protein.

In the context of the present invention, the term “protein” refers to both proteins derived from a source of protein, to peptides and to free amino acids in general. There can be one or several proteins.

In an embodiment of the invention, protein, if present, is made of whey proteins.

In another embodiment of the invention, the protein, if present, comprises lactoferrin.

The protein(s) in the protein source may be intact or hydrolysed or a combination of intact and hydrolysed proteins.

The term “intact” means in the context of the present invention proteins where the molecular structure of the protein(s) is not altered according to conventionally meaning of intact proteins. By the term “intact” is meant the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component peptides or amino acids.

The proteins may either be fully or partially hydrolysed. In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

Hydrolysation of proteins may be achieved by many means, for example by prolonged boiling in a strong acid or a strong base or by using an enzyme such as the pancreatic protease enzyme to stimulate the naturally occurring hydrolytic process.

The protein(s) according to the present invention may also be derived from free amino acids, or a combination of free amino acids and a source of protein, such as whey, lactoferrin and casein.

The whey protein may be a whey protein isolate, acid whey, sweet whey or sweet whey from which the caseino-glycomacropeptide has been removed (modified sweet whey).

Preferably, however, the whey protein is modified sweet whey.

Carbohydrates:

The composition according to the present invention can also contain a carbohydrate source, preferably as prebiotics, or in addition to prebiotics. Any carbohydrate source conventionally found in infant formulae such as lactose, saccharose, maltodextrin, starch and mixtures thereof may be used although the preferred source of carbohydrates is lactose.

The composition may comprise one or more carbohydrate.

In an embodiment of the invention, the nutritional composition, for example a human milk fortifier, comprises up to 40% carbohydrate of the caloric content. In a particular embodiment of the invention, the composition comprises up to 35% carbohydrate, such as up to 300% carbohydrate, based on the caloric content.

In another embodiment of the invention, the composition is free of carbohydrate. By “free” it is hereby meant that the composition may comprise traceable amounts of carbohydrates, such as less than 1% carbohydrate.

Non limiting examples of carbohydrates include lactose, saccharose, maltodexirin, starch, and combinations thereof.

### Probiotics:

The nutritional composition according to the present invention, for example the synthetic nutritional composition, may optionally comprise other compounds which may have a beneficial effect such as probiotics (like probiotic bacteria) in the amounts customarily found in nutritional compositions to be fed to infants.

Strains of Lactobacillus are the most common microbes employed as probiotics. However, other probiotic strains than Lactobacillus may be used in the present nutritional composition, for example the synthetic nutritional composition, for example Bifidobacterium and certain yeasts and bacilli.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. Probiotic bacteria are bacteria which have a beneficial effect on the intestinal system of humans and other animals.

In some specific embodiments, it is particularly Bifidobacteria and/or Lactobacilli.

A probiotic is a microbial cell preparation or components of microbial cells with a beneficial effect on the health or well-being of the host.

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM I-2618), Bifidobacterium longum NCC490 (CNCM I-2170), Bifidobacterium lactis NCC2818 (CNCM I-3446), Bifidobacterium breve strain A, Lactobacillus paracasei NCC2461 (CNCM I-2116), Lactobacillus johnsonii NCC533 (CNCM I-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

In an embodiment of the invention, the infant formula further includes a probiotic strain such as a probiotic bacterial strain in an amount of from 10⁶ to 10¹¹ cfu/g of composition (dry weight).

In an embodiment of the invention, the composition further comprises one or more probiotic.

### Prebiotics:

In one embodiment, the nutritional composition according to the present invention comprises one or more prebiotic. In one embodiment, the synthetic nutritional composition according to the present invention comprises one or more prebiotic.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416 which is incorporated herein by reference.

Emulsifiers

If necessary, the nutritional composition according to present invention, for example the synthetic nutritional composition, may comprise emulsifiers and/or stabilizers such as lecithin, citric esters of mono- and diglycerides, monoglycerides, diglycerides and the like. This is especially the case if the composition is provided as a combination of oils and an aqueous liquid, e.g. as an emulsion.

### Additional ingredients:

The nutritional composition of the present invention, for example the synthetic nutritional composition, may also optionally comprise other substances which may have a beneficial effect such as nucleotides, nucleosides, and the like in the amount customarily found in nutritional compositions to be fed to infants.

Other optional ingredients may be ones normally known for use on food and nutritional products, in particular infant formulas or infant formula fortifiers, provided that such optional materials are compatible with the essential components described herein, are safe and effective for their intended se, and do not otherwise unduly impair product performance.

Non-limiting examples of such optional ingredients include preservatives, anti-oxidants, buffers, colorants, flavours, thickening agents, stabilizers, and other excipients or processing aids.

Preparation

The composition according to the present invention may be prepared in any suitable manner. For example, a composition may be prepared by blending together the ingredients, such as lipid, protein and/or carbohydrate in appropriate proportions. If used, emulsifiers may be included in the blend at this stage. The vitamins and minerals may be added at this stage but are usually added later to avoid thermal degradation. Any lipophilic vitamins, such as vitamin A, D, E and K, and emulsifiers may be dissolved into the fat source prior to blending. Water, preferably water which has been subjected to reverse osmosis, may then be mixed in to a liquid mixture.

The mixture may then be thermally treated to reduce bacterial loads. Any heat sensitive components, such as vitamins and minerals may be added after heat treatment.

Further Embodiments
a) Synthetic Nutritional composition comprising one or more medium chain fatty acid (MCFA) derivative for use in promoting and/or supporting and/or optimizing brain and/or cognitive functions' development in a subject.
b) Synthetic nutritional composition for use according to embodiment a) wherein the subject is a human infant or child, and preferably a formula fed human infant or child.
c) Synthetic nutritional composition for use according to embodiment a) or b) wherein one or more MCFA derivative are provided in the form of TAGS.
d) Synthetic nutritional composition for use according to embodiment a), b) or c) wherein such composition promotes and/or supports and/or optimizes de novo biosynthesis of long chain saturated and/or long chain monounsaturated fatty acid derivatives.
e) Synthetic nutritional composition for use according to anyone of embodiments a) to d) wherein such composition promotes brain de novo myelination.
f) Synthetic nutritional composition for use according to anyone of embodiments a) to e) wherein in the subject is an infant who was born preterm or with low-birth weight (LBW) or who experienced intra-uterine growth retardation (IUGR).
g) Synthetic nutritional composition for use according to anyone of embodiments a) to f) which is a human milk fortifier.
h) Synthetic nutritional composition for use according to embodiment g) which comprises 5 to 40% w/w fatty acid derivatives, wherein 40 to 80% w/w are constituted by MCFA derivatives.
i) Synthetic nutritional composition for use according to embodiment g) or h) which comprises 5 to 30% w/w fatty acid derivatives, wherein 50 to 75% w/w are constituted by MCFA derivatives, 20 to 50% w/w protein and 15 to 40% w/w carbohydrates.
j) Synthetic nutritional composition for use according to anyone of embodiments a) to f) which is a preterm formula.
k) Synthetic nutritional composition for use according embodiment j) which comprises MCFA derivatives in amount ranging from ranging from 0.1 to 25% w/w.
l) Synthetic nutritional composition for use according to embodiment j) or k) which comprises fatty acid derivatives in amount ranging from 10 to 40% w/w, MCFA derivatives in amount ranging from 0.1 to 25% w/w, 5 to 50% w/w protein and 10 to 80% w/w carbohydrates.
m) Synthetic nutritional composition according to any of the preceding embodiment, which is a synthetic nutritional composition.
n) Use of one or more MCFA derivatives for the manufacture of a synthetic nutritional composition, for example a synthetic nutritional composition as described in embodiment g) to i) or j) to l), for promoting and/or supporting and/or optimizing brain and/or cognitive functions' development in a subject.
o) Method for promoting and/or supporting and/or optimizing brain and/or cognitive functions' development in an subject in need thereof comprising administering to such subject a synthetic nutritional composition, for example a synthetic nutritional composition as described in embodiment g) to i) or j) to l), comprising one or more medium chain fatty acids (MCFA) derivative.
p) Synthetic nutritional composition for use according to anyone of the preceding embodiments wherein the brain and/or cognitive functions' development which is promoted and/or supported and/or optimized is selected in the group consisting of: de novo myelination; brain structure, in particular the amount and spatial distribution of myelinated matter throughout the brain, and/or in specific brain regions; brain connectivity; intellectual potential; cognition; cognitive potential; learning potential; cognitive functioning; cognitive skills and abilities; cognitive functioning; and learning.
q) Use according to embodiment n) wherein the brain and/or cognitive functions' development which is promoted and/or supported and/or optimized is selected in the group consisting of: de novo myelination; brain structure, in particular the amount and spatial distribution of myelinated matter throughout the brain, and/or in specific brain regions; brain connectivity; intellectual potential; cognition; cognitive potential; learning potential; cognitive functioning; cognitive skills and abilities; cognitive functioning; and learning.
r) Method according to embodiment o) wherein the brain and/or cognitive functions' development which is promoted and/or supported and/or optimized is selected in the group consisting of: de novo myelination; brain structure, in particular the amount and spatial distribution of myelinated matter throughout the brain, and/or in specific brain regions; brain connectivity; intellectual potential; cognition; cognitive potential; learning potential; cognitive functioning; cognitive skills and abilities; cognitive functioning; and learning.
s) use of a nutritional composition, for example a synthetic nutritional composition as described in embodiment g) to i) or j) to l), comprising one or more medium chain fatty acids (MCFA) derivative to promote and/or support and/or optimize de novo biosynthesis of long chain saturated and/or long chain monounsaturated fatty acids.
t) use of a nutritional composition, for example a synthetic nutritional composition as described in embodiment g) to i) or j) to l), comprising one or more medium chain fatty acids (MCFA) derivative to promote and/or support and/or optimize brain de novo myelination.
u) use of a nutritional compositions according to the present invention, for example a synthetic nutritional composition as described in embodiment g) to i) or j) to l), is provided to promote and/or support and/or optimizing brain and/or cognitive functions' development via promotion and/or support and/or optimization of de novo biosynthesis of long chain saturated and/or long chain monounsaturated fatty acid derivatives and/or via promotion and/or support and/or optimization of brain de novo myelination.
v) use of a nutritional compositions according to the present invention, for example a synthetic nutritional composition as described in embodiment g) to i) or j) to l), is provided to promote and/or support and/or optimizing brain and/or cognitive functions' development via promotion and/or support and/or optimization of de novo biosynthesis of long chain saturated and/or long chain monounsaturated fatty acid derivatives and/or via promotion and/or support and/or optimization of brain de novo myelination, in an infant who was born preterm or with low-birth weight (LBW) or who experienced intra-uterine growth retardation (IUGR).

### Experimental Section

### EXAMPLE 1

The impact of the fortification of human milk with a lipid composition comprising according to the invention was tested in premature infants using a lipid free human milk fortifier as a control. The human milk fortifier containing lipids is abbreviated as nHMF and the control human milk fortifier which do not contain added lipids as cHMF.

Methodology

Study Design and Composition of the Control and New Human Milk Fortifiers

A total of 156 infants were screened, with 153 infants enrolled and randomized. Three subjects were screened without informed consent from the parent/guardian and were considered a screening failure. Infants were randomly assigned to either the nHMF (n=77) or cHMF (n=76) groups. Three subjects were excluded from the intent-to-treat population (n=150) due to violations of exclusion criteria determined during data review (e.g., having history of systemic disease, being born small-for-gestational age). Eleven subjects were excluded from the per-protocol population (n=139) due to occurrence of serious adverse events or non-compliance with minimum milk volume intake of 100 mL/kg body weight/d. A total of 28 infants were discontinued from the study prior to reaching W40CA due to adverse events, withdrawal by the investigator or parent (e.g., relocation of infant's family to another area, start of formula feeding), or other general reasons (e.g., parental decision, breastfeeding initiation, or hospital discharge).

This was a controlled, double-blind, randomized, parallel group clinical trial conducted at a total of 11 sites in France, Belgium, Germany, Switzerland, and Italy. Clinically stable male and female preterm infants with gestational age ≤32 wks or birth weight ≤1500 g and born to mothers who had elected to provide breast milk were enrolled in the study. Infants were excluded if they had a history of or current systemic, metabolic, or chromosomic disease, any congenital anomalies of the gastrointestinal (GI) tract, if they were small for gestational age (defined in this study as body weight 5th percentile according to Fenton [19]), or if they were receiving steroids or formula during the study period. The study was reviewed and approved by an Institutional Review Board/Independent Ethics Committee at each study site. Each subject's parent/legal representative provided their written informed consent before participating in the study. Infants tolerating ≥100 mL/kg/d of HM for >24 hrs were randomized to receive one of two powdered HM fortifiers for a minimum of 21 days. The two fortifiers provided similar energy supplementation (17 kcal/100 mL HM). For every 100 mL of HM, the new fortifier (nHMF) provided 1.4 g partially hydrolyzed whey protein, 0.7 g lipids (as medium chain triglycerides and docosahexaenoic acid), 1.5 g carbohydrate (as maltodextrin), with a blend of micronutrients. The control fortifier (cHMF) was commercially marketed at the start of the trial (FM85 Human Milk Supplement, Nesté, Switzerland), and provided 1.0 g extensively hydrolyzed whey protein, no lipids, 3.3 g carbohydrate (as lactose and maltodextrin), with a blend of micronutrients. The nHMF contained higher concentrations of vitamins A, D, E, K, B6, B12, thiamin, niacin, pantothenic acid, magnesium, iron, zinc, manganese, copper, selenium, sodium, potassium, chloride, choline, inositol, taurine, and carnitine compared to cHMF, but both fortifiers contained identical levels of calcium and phosphorus. The estimated composition of preterm HM (20) fortified with each fortifier is presented in Table 1 herebelow. Fortifiers were fed initially beginning at half-strength (Fortification Strength Increase day 1; FSI1), then advanced per hospital practice, with full-strength fortification occurring once infants could maintain intakes of 150-180 mL/kg/d (i.e. full enteral feeds; study day 1 [D1]).

TABLE 1
Nutritional composition of the control (cHMF) and new human milk fortifier
(nHMF) used in the present study

| Nutrients (per 100 g of powder) | cHMF | nHMF |
|---|---|---|
| Protein (g) | 20.00 | 35.50 |
| Carbohydrates (g) | 66.00 | 32.40 |
| Lipid content (g) | 0.38 | 18.10 |
| Saturated fatty acids (g) | — | 12.20 |
| Medium chain fatty acids (MCFA, g) | — | 12.50 |
| Linoleic acid (LA, mg) | — | 958.00 |
| α-Linolenic acid (ALA, mg) | — | 417.00 |
| Docosahexaenoic acid (DHA, mg) | — | 157.00 |

Blood Collection

Blood (0.7 mL) was collected in EDTA-containing vaccutainers from the infant within D1 and again on D21 of life. The blood was immediately centrifuged for 10 min at 1300×g, and plasma and RBC were stored in microtubes at −80° C. until analysis.

Plasma Lipid Classes Separation and Fatty Acid Methyl Esters (FAME) Preparation Plasma lipids were extracted from plasma according to Folch et al. “A simple method for the isolation and purification of total lipids and from animal tissues”, J. Biol. Chem. 1957, 226:497-509.he lipid classes were separated by thin-layer chromatography (TLC) and sample migration was performed with hexane/diethyl ether/acetic acid (80/20/1; v/v/v). After drying the lipid classes were visualized by spraying the TLC plate with 1,2-dichlorofluorescein and detected under UV-light. The lipid fractions (PL and TAG) were identified by comparison with standards and were scraped-off to be collected in glass tubes. Standard trimyristoleine and diheptadecanoyl-sn-glycero-3-phosphoethanolamine were added to the TAG and PL extracts, respectively. Fatty acids in plasma TAG and PL were transesterified according to the method of Morrison and Smith, “Preparation of fatty acid methyl esters and dimethylacetals from lipids with boronfluoride-methanol” Journal of Lipid Research, vol. 5, 1964.

RBC Phospholipid Classes Separation and Fatty Acid Methyl Esters (FAME) Preparation

Lipids were extracted from the RBC according to the method of Peuchant et al., “One-step extraction of human erythrocyte lipids allowing rapid determination of fatty acid composition”, Analytical Biochemistry, v: 181 1:2 p: 341-4, 1989C and PE were separated from RBC lipid extract by TLC and sample migration was performed with chloroform/methanol/acetic acid/water (50/37.5/3.5/2; vol/vol/vol) as migration solvent. PC and PE were visualized by spraying the TLC with 1,2-dichlorofluorescein and detected under UV-light. The lipid fractions from PC and PE were identified by comparison with standards and were scraped-off and collected in glass tubes. Standard diheptadecanoyl-sn-glycero-3-phosphoethanolamine and -phosphatidylcholine were added to the PE and PC extracts, respectively. FAME were obtained as described for plasma TAG and PL fractions.

Fatty Acid Methyl Esters (FAME) Analysis

FAME were analyzed by gas-liquid chromatograpy (GLC) on a BPX 70 capillary column (60 m long, 0.25 μm film, 0.25 mm i.d., SGE, hydrogen as carrier gas, split ratio of 1:80). The GLC system consisted of a gas chromatograph Focus GS (Thermofinnigan, Courtaboeuf, France) equipped with a flame-ionization detector maintained at 250° C. The injector temperature was 250° C. The column temperature was increased from 150° C. to 200° C. (1.3° C/min), maintained at 200° C. for 20 min, increased from 200° C. to 235° C. (10° C./min), and held at 235° C. for 20 min. Data handling was performed using Chromquest software (Thermofinnigan, Courtaboeuf, France). Pure FAME mixture (Sigma, St Louis Mo., USA) of known composition were used as standard for column calibration. The variation in peak area between injections was less than 2%.

Statistical Analysis

Several fatty acids have been measured at day 1 (visit 1, day of full fortification) and at day 21 after full fortification in plasma total phospholipids (plasma PL), plasma TAG, RBC PC and RBC PE. For each fatty acid and for each compartment (PL plasma, TG plasma, RBC PC and RBC PE), summary statistics at each visit have been calculated. Data were nearly log-normal distributed, geometric mean and geometric standard deviation are provided in the present report instead of arithmetic mean and standard deviation. Fatty acid relative concentration was analysed at visit 5 (log-transformation) using a (mixed-effect) ANCOVA model adjusts for gestational age at day 1, weight at day 1, fatty acid concentration at day 1, gender, center and treatment group (with center considered as random effect).

Results

At the end of the treatment period (day 21), the fatty acid composition of circulating lipids and in particular plasma triacylglycerols (plasma TAG), plasma phospholipids (plasma PL), red blood cells phosphatidylcholine (RBC-PC) and phosphatidylethanolamine (RBC-PE) were analyzed. Phosphatidylethanolamine fatty acid composition from RCB-PE compartment, a recognized marker in human of the fatty acid metabolism and accumulation in tissues and especially brain [see Innis S, “n-3 fatty acids requirements in the newborn”, LIPIDS, Vol. 27, no. 11 (1992) and Sauerwald U. et Al, “Effect of different levels of DHA supply on Fatty Acid status and LA and ALA conversion on Preterm infants, vol 4, number 3, March 2012], provide evidences of the increase levels of monounsaturated fatty acids (Table 2) and in particular the increase of erucic (22:1 n-9), gondoic acid (20:1 n-9) and palmitic acid (18:1 n-7) (Table 3).

TABLE 2
ANCOVA model for the log of the sum of monounsaturated fatty acids (MUFA)
concentration at day 21 (visit 5) in plasma phospholipids (plasma PL), plasma
triacylglycerols (plasma TAG), red blood cells phosphatidylcholine (RBC-PC) and red blood
cells phosphatidylethanolamine (RBC-PE) with gestational age at day 1, weight at day 1,
log of the S AGMI concentration at visit 1, gender and treatment group as covariates,
center considered as a random effect-Estimates, standard errors, 95% confidence
intervals for treatment effect are displayed. Two-sided p-value is given for the treatment
effect (ITT analysis set)

| | ESTIMATE STD. | ERROR | 2.50% | 97.50% | P-VALUE |
|---|---|---|---|---|---|
| PLASMA PL | 0.074 | 0.046 | −0.02 | 0.169 | 0.12 |
| PLASMA TAG | −0.006 | 0.024 | −0.055 | 0.043 | 0.808 |
| RBC-PC | 0.069 | 0.034 | 0.000 | 0.139 | 0.051 |
| RBC-PE | 0.044 | 0.018 | 0.007 | 0.081 | 0.023 |

TABLE 3
Fatty acid profile (in g per 100 g of fatty acids) of red blood cells
phosphatidylethanolamine (RBC-PE) in preterm infants receiving human milk
fortified with a control (cHMF) or with a new human milk fortifier (nHMF)
at the beginning of the study and after 21 days of treatment.

| | Baseline | | After 21 days | | | |
|---|---|---|---|---|---|---|
| | cHMF | nHMF | cHMF | nHMF | estimate | p value |
| 14:0 | 0.10 | 0.14 | 0.17 | 0.18 | −0.056 | |
| 15:0 | 0.22 | 0.31 | 0.32 | 0.31 | −0.611 | 0.024 |
| 16:0 | 15.30 | 16.37 | 16.47 | 15.71 | −0.123 | 0.040 |
| 16:0 DMA | 5.24 | 5.38 | 5.42 | 5.61 | 0.061 | |
| 16:1 n-7 | 0.34 | 0.36 | 0.39 | 0.41 | −0.039 | |
| 16:1 n-9 | 0.31 | 0.34 | 0.36 | 0.38 | −0.061 | |
| 18:0 | 6.69 | 6.56 | 6.84 | 6.64 | −0.019 | |
| 18:0 DMA | 9.30 | 9.28 | 8.62 | 8.45 | 0.007 | |
| 18:1 DMA | 3.77 | 3.37 | 3.55 | 3.54 | 0.094 | |
| 18:1 n-7 | 1.25 | 1.15 | 1.29 | 1.40 | 0.114 | 0.013 |
| 18:1 n-9 | 15.49 | 14.65 | 14.31 | 14.67 | 0.029 | |
| trans-18:1 | 0.22 | 0.22 | 0.29 | 0.26 | −0.074 | |
| 18:2 n-6 (LA) | 2.66 | 2.85 | 2.97 | 3.30 | 0.013 | |
| 18:3 n-3 (ALA) | 0.20 | 0.25 | 0.25 | 0.29 | −0.139 | |
| 18:3 n-6 (GLA) | 0.09 | 0.09 | 0.09 | 0.11 | 0.118 | |
| 20:0 | 0.09 | 0.09 | 0.09 | 0.10 | 0.059 | |
| 20:1 n-7 | 0.06 | 0.06 | 0.08 | 0.09 | −0.011 | |
| 20:1 n-9 | 0.50 | 0.43 | 0.56 | 0.60 | 0.174 | 0.003 |
| 20:2 n-6 | 0.17 | 0.17 | 0.21 | 0.20 | 0.012 | |
| 20:3 n-6 (DGLA) | 1.76 | 1.56 | 1.80 | 1.84 | 0.099 | 0.031 |
| 20:3 n-9 | 1.33 | 1.22 | 1.58 | 1.95 | 0.247 | 0.011 |
| 20:4 n-6 (ARA) | 20.78 | 21.13 | 20.54 | 19.65 | −0.024 | |
| 20:5 n-3 (EPA) | 0.79 | 0.71 | 0.68 | 0.95 | 0.301 | <0.001 |
| 22:0 | 0.03 | 0.03 | 0.04 | 0.05 | −0.250 | |
| 22:1 n-9 | 0.07 | 0.05 | 0.07 | 0.07 | 0.352 | 0.008 |
| 22:4 n-6 | 4.75 | 4.77 | 4.78 | 4.48 | −0.036 | |
| 22:5 n-3 (DPA) | 1.46 | 1.31 | 1.48 | 1.65 | 0.117 | 0.019 |
| 22:5 n-6 (DPA) | 0.97 | 0.90 | 1.00 | 0.95 | 0.047 | |
| 22:6 n-3 (DHA) | 5.64 | 5.76 | 5.14 | 5.61 | 0.092 | 0.016 |
| 24:0 | 0.05 | 0.04 | 0.04 | 0.04 | −0.201 | |
| 24:1 n-9 | 0.05 | 0.04 | 0.03 | 0.03 | 0.172 | |

According to results presented in Table 3, it also appears that levels of other fatty acids well known to be involved in myelination and brain development process are increased by administration of a human milk fortifier according to the invention [such as for example 22:5 n-3 (DPA), 20:5 n-3 (EPA) and 22:6 n-3 (DHA)].

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

### EXAMPLE 2

Co Culture of Neurons and Oligodendrocytes (OL)

Neurons/Oligodendrocytes were cultured as previously described by Charles et al., 2000. Pregnant female rats of 17 days gestation were killed by cervical dislocation (Rats Wistar) and the foetuses removed from the uterus. The Forebrains were removed and placed in ice-cold medium of Leibovitz (L15) containing 2% of Penicillin-Streptomycin (PS) and 1% of bovine serum albumin (BSA). Forebrains were dissociated by trypsinisation for 20 min at 37²C (Trypsin EDTA 1X). The reaction was stopped by the addition of Dulbecco's modified Eagle's medium (DMEM) containing DNAase I grade II (0.1 mg/ml) and 10% of foetal calf serum (FCS). Cells were then mechanically dissociated by 3 passages through a 10 ml pipette. Cells were then centrifuged at 180 x g for 10 min at 4° C. temperature on a layer of BSA (3.5%) in L15 medium. The supernatant was discarded and the cells of pellet were re-suspended in DMEM containing 10% of FCS. Cells were then centrifuged at 515×g for 10 min at 4° C. The supernatant was discarded and the cells of pellet were re-suspended in a culture medium consisting of Neurobasal supplemented with 2% of B27, 2 mM of L-glutamine (L Glu), 2% of PS solution, 1% of FCS and 10 ng/ml of platelet-derived growth factor (PDGF-AA). Viable cells were counted in a Neubauer cytometer using the trypan blue exclusion test. The cells were seeded at a density of 20000 cells/well in 96 well-plates pre-coated with poly-L-lysine and laminin.

The day following seeding (day 1 of culture), cells were incubated with a test compound (selected from those listed in table 3), or estradiol. Control cells were not incubated with a test compound or estradiol. Estradiol was used as positive control. Estradiol is known to induce OPC (Oligo dendrocytes precursors cells) proliferation. The positive effect of estradiol on OL differentiation has also been demonstrated, as has its effect on the early myelination process. The positive effect of estradiol on neurite outgrowth was also published (for review see Alevaro et al., 2010).

The plates were maintained at 37° C. in a humidified incubator, in an atmosphere of air (95%)-CO2 (5%). Half of the medium was replaced every other day with fresh medium and test compound or control compound. The test or control compounds were maintained at the defined concentration for the duration of the experiments. Compounds were tested on 1 culture (6 wells per conditions). Cells were then used on day 12, 18 or 30 of culture to measure one of either proliferation of OPC, differentiation of OPC into OL and early myelination process (myelin wrapping), or maturation of OL (myelin maturation) and mature myelination process (myelin wrapping).

Proliferation of OPC—Measurement of A2B5 Positive Cells and Total Axonal Length (NF)

On day 12 of culture, cells were fixed by a cold mixture of absolute ethanol (95%) and pure acetic acid (5%) for 5 min. The cells were then permeabilized and non-specific sites were blocked with a solution of phosphate buffered saline (PBS) containing 0.1% of saponin and 1% FCS for 15 min at room temperature.

Cells were then incubated with Monoclonal Anti-A2B5 conjugated alexa fluor® 488 produced in mouse at dilution of 1/200 in PBS containing 1% FCS, 0.1% saponin, for 2 h at room temperature and with anti-NF (Neurofilament 200 phosphorylated and non-phosphorylated) produced in rabbit at dilution of 1/500 in PBS containing 1% FCS, 0.1% saponin for 2 h at room temperature. This antibody was revealed with Alexa Fluor 568 goat anti-rabbit at the dilution of 1/400 in PBS with 1% FCS, 0.1% saponin, for 1 h at room temperature.

The total number of OPC (number of A2B5 positive cells) was quantified (to evaluate the proliferation), the axonal network was measured (total axonal length (NF)) to assess the effect of the compound on the neuronal network (the quality of the myelination is directly linked to the quality of the axonal network).

Differentiation of OPC into OL and Myelination Process (Myelin Wrapping)—Measurement of Number and Area of MAG Positive Cells, Overlap MAG/NF Wrapping, and Total Axonal Length (NF)

On day 18 of culture, cells were fixed by a cold mixture of absolute ethanol (95%) and pure acetic acid (5%) for 5 min. The cells were then permeabilized and non-specific sites were blocked with a solution of phosphate buffered saline (PBS) containing 0.1% of saponin and 1% FCS for 15 min at room temperature.

Cells were then incubated with Monoclonal Anti-MAG produced in mouse at dilution of 1/400 in PBS containing 1% FCS, 0.1% saponin, and with anti-NF (Neurofilament 200 phosphorylated and non-phosphorylated) produced in rabbit at dilution of 1/500 in PBS containing 1% FCS, 0.1% saponin for 2 h at room temperature. These antibodies were revealed with CF 488 A goat anti-mouse at the dilution of 1/800 in PBS with 1% FCS, 0.1% saponin and Alexa Fluor 568 goat anti-rabbit at the dilution of 1/800 in PBS with 1% FCS, 0.1% saponin, for 1 h at room temperature.

The total number of OL was quantified (number and area of MAG positive cells) (to evaluate the differentiation process), as well as the wrapping of OPC around axons (overlap MAG/NF wrapping) (myelination process). The axonal network was measured (total axonal length (NF) to assess the effect of the compounds on the neuronal network.

Maturation of OL (Myelin Maturation)—Measurement of Number and Area of MBP Positive Cells, Overlap MBP/NF Wrapping, and Total Axonal Length (NF)

On day 30 of culture, cells were fixed by a cold mixture of absolute ethanol (95%) and pure acetic acid (5%) for 5 min. The cells were then permeabilized and non-specific sites were blocked with a solution of phosphate buffered saline (PBS) containing 0.1% of saponin and 1% FCS for 15 min at room temperature.

Cells were then incubated with Monoclonal Anti-MBP produced in mouse at dilution of 1/1000 in PBS containing 1% FCS, 0.1% saponin, and with anti-NF (Neurofilament 200 phosphorylated and non-phosphorylated) produced in rabbit at dilution of 1/500 in PBS containing 1% FCS, 0.1% saponin for 2 h at room temperature. These antibodies were revealed with CF 488 A goat anti-mouse at the dilution of 1/800 in PBS with 1% FCS, 0.1% saponin and Alexa Fluor 568 goat anti-rabbit at the dilution of 1/400 in PBS with 1% FCS, 0.1% saponin, for 1 h at room temperature.

The total number of OL was assessed (number and area of MBP positive cells) (to evaluate the OL maturation) as well as the wrapping of myelin around axon (overlap MBP/NF(wrapping)). The axonal network was measured (Total axonal length (NF)) to assess the effect of the compounds on the neuronal network.

For all measurements, once the culture was done (6 wells per conditions). For each condition tested, 30 pictures (each picture representing a field) per well were taken and analyzed using ImageXpress (Molecular devices) with 20× magnification equipped with LED lamp (excitation 360/480/565 and emission 460/535/620). The 30 pictures were automatically taken and represented 80% of the total surface of the culture well. Results were expressed in terms of cumulated mean length in μm of neurite network, or myelin sheath labeled for a given marker (MAG or MBP) per field. The overlapping area between NF and MAG or MBP was measured to evaluate the wrapping.

To assess OPC population, MAG positive cell population, MBP positive cell population, an automatic counting of number of positive cells per picture (=field) was done. The results were expressed in mean number of positive cells per field.

All the images were taken under the same conditions.

TABLE 3
PLATE 1 (A2B5/NF)
Control
Estradiol (150 nM)
DHA (0.15 μM)
DHA (1.5 μM)
Stearic acid (SA) (50 μM)
Stearic acid (SA) (5 μM)
Stearic acid (SA) (0.5 μM)

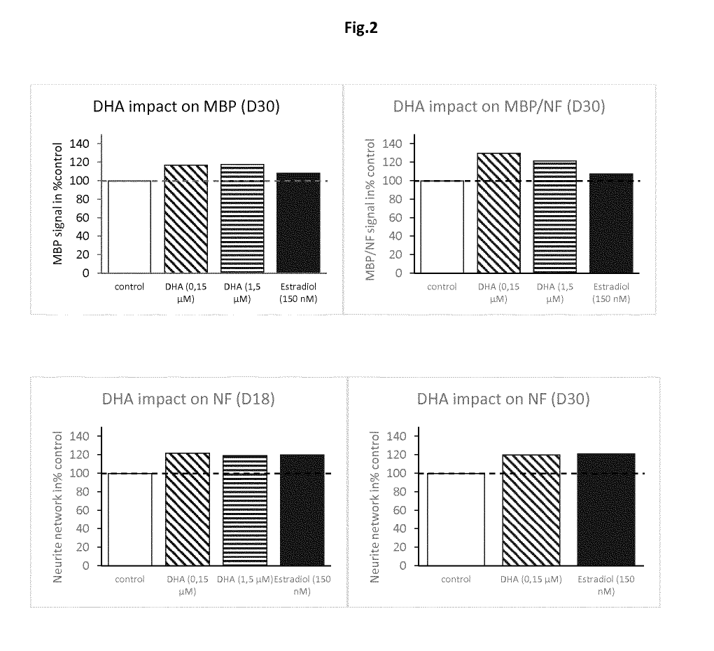

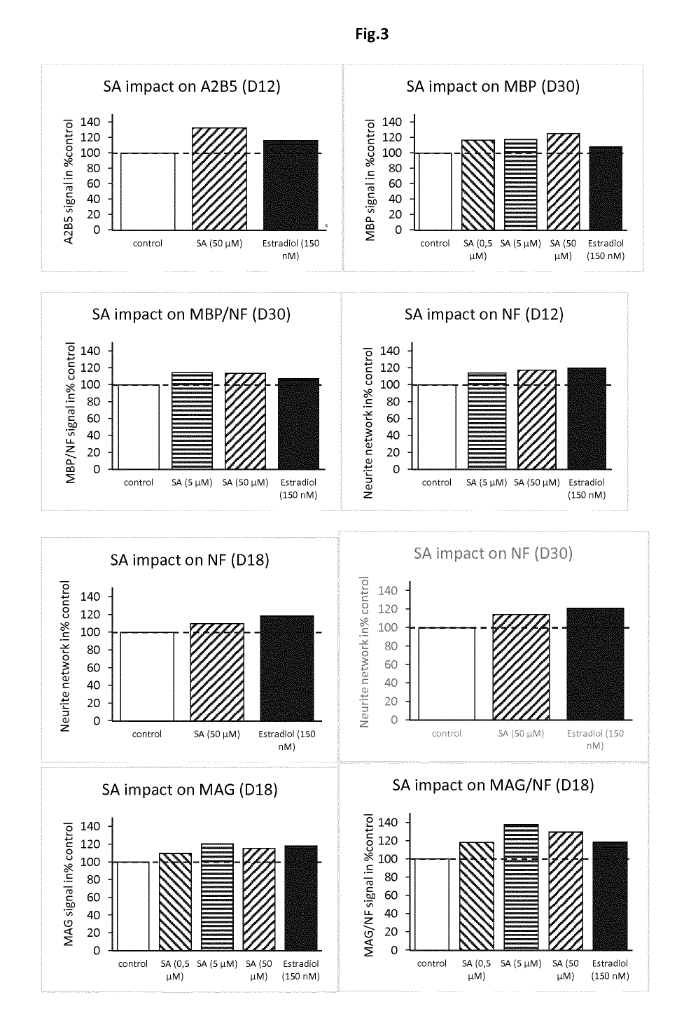

Results are show in FIGS. 2 and 3

### EXAMPLE 3

Materials and Methods

1. Feeder Layer Preparation: Dissociation of Neonatal Cortices and Maintenance of Mixed Glial Cultures

Freshly dissected brains were added to a 37° C. water bath for 3 min, then cortices were diced through a P1000 pipette tip to generate smaller fragments. 75 μL of OPC papain solution per brain were added, then tissues were incubated in a 37° C. water bath for 20 min. The tissue suspension was then additioned with mixed glial culture in order to allow inactivation of the OPC papain solution.

Tissue were subsequently triturated using a sterile flame-polished glass Pasteur pipette, then 4 mL of mixed glial culture media per brain was added. Cells were centrifuged at 1200 rpm (˜300 g) for 5 min, then cells were resuspended in warm mixed glial culture media and plated into PLL-coated flask.

4 hours following plating, a full media change was performed in order to remove much of the debris caused by the trituration, and promote culture viability. After 3 days of culture, a ⅔ media change was performed, and no subsequent medium change was performed. Cells were then maintained in culture until confluency.

2. Hippocampal Neurons Preparation

Hippocampal neurons were isolated from embryonic (E18) pups of Sprague Dawley rats. Briefly, following animal sacrifice, brains were isolated, meninges removed from the medial aspect of the cerebral hemispheres, then hippocampi dissected out and kept at 4° C. until process completion.

Tissue were then incubated with 2.5% trypsin for 15 min in a water bath at 37° C., then gently washed and kept in culturing media. Hippocampal dissociation was performed by repeatedly pipetting them up and down with a functionalized sterile Pasteur pipette.

Following mechanical dissociation, cells were plated at desired density in neuronal plating medium, let recover for 4 hours, then put in compete neuronal culturing medium.

3. Purification of OPCs from Mixed Glial Cultures for Establishment of OL/Hippocampal Neurons o-Cultures

On Day 9 of the mixed glial culture, flasks were shaken at 50 rpm for 45 min on an orbital shaker in a 5% CO2 tissue culture incubator. The purpose of this shake was to remove any loosely adherent contaminating cells from the monolayer.

Media was then changed and replaced with 4 mL of fresh mixed glial culture media supplemented with 5 μg/mL insulin. Flasks were then repositioned onto the shaker, equilibrated for approximately 3 hours, then shaken for approximately 16 hours at 220 rpm (overnight).

The next morning, mixed glia culture medium containing microglia and OPCs cells were collected and pre-plated on P100 petri dish (not treated for culture) for 30 minutes in order to purify OPCs cells; microglia cells start immediately to adhere to petri while OPCs cells remained in the surnatant medium.

After 30 minutes of pre-plate, medium was collected and OLs were counted and seeded on hippocampal neurons in a final volume of 1 mL OL media.

A full OL media (minus CNTF) change was performed, then cells were maintained in culture until the appropriate experimental timings.

For maturation experiments, the experimental procedure was as follows:
a. Growth of OPCs on feeder layer of astrocytes for 10 DIV
b. Isolation of OPCs (Day 0)
c. Administration of compounds (Day 3)
d. Quantitative evaluation of maturation at Day 4, 7 and 10.

For myelination experiments, the experimental procedure was as follows:
a. Growth of hippocampal neurons until complete neuronal network maturation (14 DIV)
b. Concomitant growth of OPCs on feeder layer of astrocytes for 10 DIV
c. Isolation of OPCs and coculturing with neurons (Day 14)
d. Administration of compounds (Day 15)
e. Quantitative evaluation of myelination at Day 15 (1 day after coculture plating, before compound treatment), 18, 21/23 and 28/29 of coculturing

4. Acquisition of Images

All cultures at the different experimental time points, were fixed in 4% paraformaldehyde and 4% sucrose at room temperature (RT) for 10 min. Primary and secondary antibodies were applied in GDB buffer (30 mM phosphate buffer, pH 7.4, containing 0.2% gelatin, 0.5% Triton X-100, and 0.8 M NaCl) for 2 h at room temperature. cells were stained with appropriate marker (primary antibody used: Anti-A2B5 antibody (ABCAM cat. ab53521), Rat anti MBP (BIO-RAD cat. aa82-87), Oligodendrocyte Marker O4 Antibody (R&D Systems cat. MAB1326), Anti-βIII Tubulin mAb (Promega cat. G7121); secondary antibody used:

Alexa anti rat 555 (Life Tech A-21434), Alexa anti mouse 488 (Life Tech A-11009). Following immunocytochemical staining all images were acquired with Array Scan XTI (ThermoScientific); the objective was 20× at binning 2×2. For each condition and replica well (triplicate) a minimum of 15 images were taken.

For the analysis of all acquired images the HCS Studio Cell Analysis Software was used, in particular the “Scan” application.

OPC Papain Solution (made up in MEM)

Papain solution 1.54 mg/mL

L-cysteine 360 μg/mL

DNase I 60 μg/mL

Mixed Glial Culture Media (made up in DMEM)

FBS 10%

Pen/Strep (0.33% from stock) 33 units/mL Penicillin and 33 μg/mL Streptomycin GlutaMAX 1%

OL media

DMEM

100× OL-Supplement

Bovine insulin (from 1 mg/mL stock)

GlutaMAX

Holo-transferrin (from 33 mg/mL stock)

B27 Supplement

FBS

CNTF (from 50 ng/μL stock)

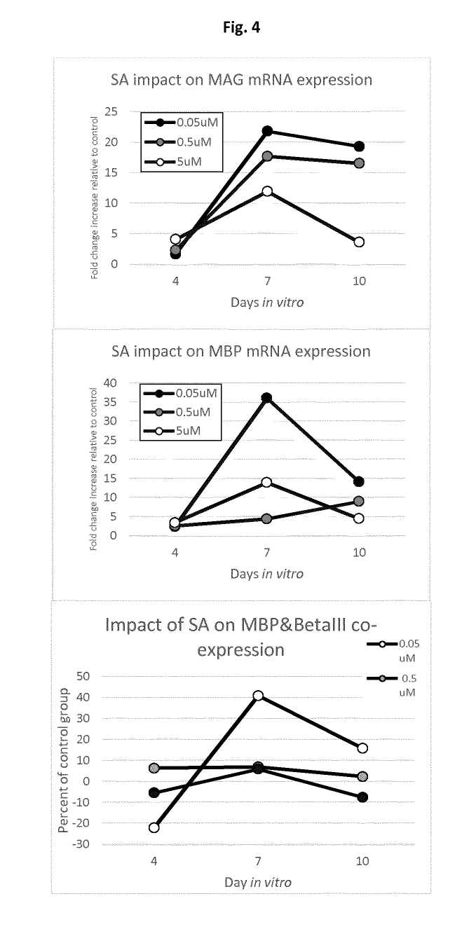

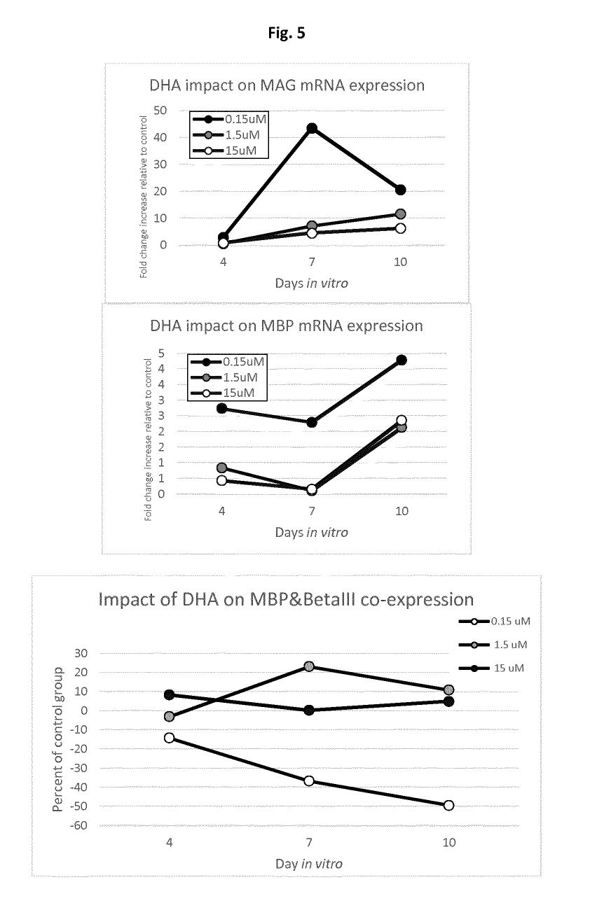

Results for stearic acid and DHA are show in FIGS. 4 and 5 respectively.

1. A method for achieving a result selected from the group consisting of promoting, supporting, optimizing brain and cognitive functions and development comprising administering a nutritional composition comprising one or more medium chain fatty acid (MCFA) derivative to a subject.

2. Method according to claim 1 wherein the subject is a human infant or child.

3. Method according to claim 1 wherein one or more MCFA derivative are provided in the form of TAGs.

4. Method according to claim 1 wherein brain and/or cognitive functions' development is achieved via promotion and/or support and/or optimization of de novo biosynthesis of long chain saturated and/or long chain monounsaturated fatty acid derivatives.

5. Method according to claim 1 wherein brain and/or cognitive functions' development is achieved via promotion of brain de novo myelination.

6. Method according to claim 1 wherein brain and/or cognitive functions' development is supported and/or promoted and/or optimized in an infant who was born preterm or with low-birth weight (LBW) or who experienced intra-uterine growth retardation (IUGR).

7. Method according to claim 1 wherein the composition is a human milk fortifier.

8. Method according to claim 7 wherein the composition comprises 5 to 40% w/w fatty acid derivatives, wherein 40 to 80% w/w are constituted by MCFA derivatives.

9. Method according to claim 7 wherein the composition comprises 5 to 30% w/w fatty acid derivatives, wherein 50 to 75% w/w are constituted by MCFA derivatives, 20 to 50% w/w protein and 15 to 40% w/w carbohydrates.

10. Method according to claim 1 wherein the composition is a preterm formula.

11. Method according to claim 10 wherein the composition comprises MCFA derivatives in amount ranging from ranging from 0.1 to 25% w/w.

12. Method according to claim 10 wherein the composition comprises fatty acid derivatives in amount ranging from 10 to 40% w/w, MCFA derivatives in amount ranging from 0.1 to 25% w/w, 5 to 50% w/w protein and 10 to 80% w/w carbohydrates.

13. Method according to claim 1, which wherein the composition is a synthetic nutritional composition.

14. (canceled)

15. Method for promoting and/or supporting and/or optimizing brain and/or cognitive functions' development in an a formula fed human subject in need thereof comprising administering to such subject a nutritional composition comprising one or more medium chain fatty acids (MCFA) derivative.

16. (canceled)
